(19) United States
(12) Patent Application Publication
(10) Pub. No.: US 2025/0075173 A1
(43) Pub. Date: Mar. 6, 2025

(54) Acellular Substrates and Methods of Making the Same

(71) Applicant: Ronawk, Inc., Olathe, KS (US)

(72) Inventor: Adam J. Mellott, Parkville, MO (US)

(21) Appl. No.: 18/726,679

(22) PCT Filed: Jan. 4, 2023
(86) PCT No.: PCT/US2023/010091

§ 371 Date: Jul. 3, 2024

Related U.S. Application Data
(60) Provisional application No. 63/296,274, filed on Jan. 4, 2022.

(51) Int. Cl. C12N 5/00

(52) U.S. Cl. CPC C12N 5/0062; C12N 2513/00; C12N 2533/90; C12N 2539/00

(57) ABSTRACT

Acellular substrates that provide continuous 3D growth of a variety of cells and/or tissues and methods of making the same are provided. The acellular substrates have a three-dimensional (3D) macrostructure defined by a continuous matrix of extracellular matrix (ECM) material associated with a first cell type of interest and a network of microporous channels and/or chambers extending throughout the continuous matrix of ECM material associated with the first cell type of interest. Methods of forming a personalized graft using a patient's own cells is also provided.

### CROSS-REFERENCE TO RELATED APPLICATIONS

This application claims priority to U.S. Provisional Application No. 63/296,274, filed Jan. 4, 2022, which is expressly incorporated by reference herein in its entirety.

### TECHNICAL FIELD

Embodiments of the presently-disclosed invention relate generally to acellular substrates that provide continuous 3D growth of a variety of cells and/or tissues and methods of making the same. The acellular substrates have a three-dimensional (3D) macrostructure defined by a continuous matrix of extracellular matrix (ECM) material associated with a first cell type of interest and a network of microporous channels and/or chambers extending throughout the continuous matrix of ECM material associated with the first cell type of interest. Methods of forming a personalized graft using a patient's own cells is also provided.

### BACKGROUND

Connective tissues, such as bone, cartilage, skin, nerve, lung, muscle, and many others contain cells that secrete and organize extracellular matrix (ECM) proteins to create scaffolds to allow cells to migrate, proliferate, and organize to form tissues. The interplay between the organization of composition and structure of ECM is well documented. For instance, each tissue has a different composition and organizational structure to instruct cell types how to arrange, organize, and function.

There remains a need in the art for the development of scaffolds that enable the creation of biological scaffolds from any connective tissue of interest, such as an ECM scaffold (e.g., acellular substrate) that can accept autologous cells from a patient to build a personalized graft.

### SUMMARY OF INVENTION

One or more embodiments of the invention may address one or more of the aforementioned problems. Certain embodiments according to the invention provide an acellular substrate (e.g., a scaffolding) comprising a three-dimensional (3D) macrostructure defined by a continuous matrix of extracellular matrix (ECM) material associated with a first cell type of interest and a network of microporous channels and/or chambers extending throughout the continuous matrix of ECM material associated with the first cell type of interest. The 3D macrostructure may comprise a top surface, a bottom surface, and a thickness defined by at least one side edge extending from the top surface to the bottom surface.

In another aspect, certain embodiments according to the invention provide a method of forming an acellular substrate, comprising: (i) forming or placing a network of microstrands and/or micropods comprising a degradable hydrogel material within a mold; (ii) seeding the network of microstrands and/or micropods by adding an initial culture media including cells of a cell type of interest into the mold housing the network of microstrands and/or micropods; (iii) feeding the cells by perfusing fresh culture media through the mold to provide cells with nutrients until a tissue has grown and expanded to fill the mold; (iv) performing a decellularization operation on the tissue located in the mold forming a continuous matrix of ECM material associated with the cell type of interest; and (v) forming a network of microporous channels and/or chambers extending throughout the continuous matrix of ECM material associated with the cell type of interest by degrading and removing the network of microstrands and/or micropods to provide the acellular substrate.

In another aspect, certain embodiments according to the invention provide a method of forming a personalized graft, comprising: (i) providing or forming one or more acellular substrates, such as those described and disclosed herein, wherein the first cell type of interest is associated with a patient's tissue having an anomaly, such a particular organ tissue; (ii) seeding the one or more acellular substrates with healthy native cells associated with the patients tissue having an anomaly; and (iii) feeding the healthy native cells with a culture media, and allowing the healthy native cells to propagate throughout the network of microporous channels and/or chambers of the acellular substrate forming the personalized graft.

### BRIEF DESCRIPTION OF THE DRAWING(S)

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout, and wherein:

FIG. 1 illustrates an acellular substrate in accordance with certain embodiments of the invention;

FIG. 2 illustrates a cuboidal mold having a network of microchannels and/or micropods 3D printed therein or disposed therein;

FIG. 3 illustrates the seeding of a cell type of interest onto the network of microchannels and/or micropods, and growth of the extracellular matrix of the cell type of interest to fill the cuboidal mold;

FIG. 4 illustrates the decellularized tissue and subsequent degradation or dissolution of the hydrogel material to form an intermediate acellular substrate;

FIG. 5 illustrates the intermediate acellular substrate being lyophilized and subsequently removed from the cuboidal mold followed by sterilization; and

FIGS. 6-9 illustrate different configurations of a plurality of joined acellular substrates in accordance with certain embodiments of the invention.

### DETAILED DESCRIPTION

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The presently-disclosed invention relates generally to three-dimensional (3D) acellular substrates that contain the structure components and cues to instruct cells on how to behave to form tissues. In this regard, each tissue has a different composition and organizational structure to instruct cell types how to arrange, organize, and function. Accordingly, certain embodiments of the invention include acellular substrates and methods of making the same that enables the creation of biological scaffolds from any connective tissue of interest. As described in more detail below, a degradable inverse hydrogel mold may be sterilized and placed inside a cuboidal, for example, mold that may range in volume, for example, from a cubic centimeter to a cubic decimeter. To this mold, cells from specific tissues are added and cultured until a full tissue has formed by perfusing media through the mold to provide cells with nutrients. Once a full tissue has formed, cells are removed through decellularization by applying a mild detergent, dialyzing the ECM. Afterward, the ECM may be is subject to multiple freeze-thaw cycles to remove any remaining cell debris. Afterward, the ECM may be washed with a mild detergent and dialyzed again. The hydrogel inverse mold may then be dissolved, for example, via photo lysis. The ECM may then lyophilized and stored until use. The ECM scaffold (e.g., acellular substrate), for example, may be used as a building block to create specific tissues. For example, if the ECM scaffold (e.g., acellular substrate) is made from keratinocytes initially, and induced pluripotent stem cells from a patient are added to the ECM scaffold (e.g., acellular substrate), new auto epidermal grafts can be formed using the ECM scaffold (e.g., acellular substrate). In accordance with certain embodiments, for instance, the present invention provides for the use of producing a ECM scaffold (e.g., acellular substrate) that can accept autologous cells from a patient to build a personalized graft.

In accordance with certain embodiments of the invention, the acellular substrates may comprises a custom scaffold that is designed to culture cells that are used to form a custom ECM scaffold for a particular patient. For example, the cells are removed from the ECM scaffold, and the patient's own cells are added to the ECM scaffold designed for them. This means when a patient has an anomaly in a tissue, a custom mold (e.g., a 3D network of microstrands and/or micropods based on the tissue morphology of the tissue in question and formed from a hydrogel material) can be created to fix the anomaly, and create a custom scaffold (e.g., dissolution or degradation of the hydrogel material forming the 3D network of microstrands and/or micropods) personalized graft for the patient. In this regard, the microstructure of a tissue can be used to differentiate stem cells into requisite cells without the need for growth factor cocktails. This means, the design of custom scaffolds, in accordance with certain embodiments of the invention, may be provided that direct the differentiation fate of stem cells to create personalized tissue grafts for patients to correct tissue anomalies. By using magnetic resonance imaging and 3D printing, for example, the preparation of custom hydrogel molds (e.g., a 3D network of microstrands and/or micropods based on the tissue morphology of the tissue in question and formed from a hydrogel material) to create custom ECM scaffolds (e.g., acellular substrates) may be provided by certain embodiments of the invention. Previously, structure alone has never been considered enough to differentiate and control cell fate for forming a tissue. The primary mechanism behind controlling cell fate has always thought to be growth factor and signal-based. In accordance with certain embodiments of the invention, however, substrate and structure have been shown to control cell fate, which may be leveraged to craft novel scaffolds for building custom grafts to treat patient tissue anomalies.

In accordance with certain embodiments of the invention, the method of forming an acellular substrate allows for the creation of custom scaffolds from a patient's own anatomy to form grafts from the application of a patient's own cells. In this regard, for example, magnetic resonance imaging and stereolithography (3D Printing) may be used to print an inverse hydrogel scaffold mold based on a patient's own tissue for which cells can be seeded into for the purpose of forming a custom extracellular matrix scaffold. An inverse hydrogel scaffold mold, for instance, is a network of microstrands and/or micropods structured based upon tissue morphology of a cell line of interest. Moreover, the methods in accordance with certain embodiments of the invention allows for tailor-made scaffolds to be made from different formulations of extracellular matrix based on the deposition and ratio of cells seeded on the hydrogel scaffold mold. In this regard, a single acellular substrate may be seeded with multiple cell lines of interest or a one acellular substrate may be seeded with a first cell line of interest while an adjoining acellular substrate may be seeded with a second cell line of interest, and allowed to grow together and form an interface between the two cell lines of interest. The cell(s) of interest may be used to form a continuous extracellular matrix that contain individual or combinations of extracellular matrix components, such as Glycosaminoglycans (GAGs); Proteoglycans; Hyaluronic acid (HA); Heparin sulfate (HS); Chondroitin sulfate (CS); Keratan sulfate (KS); Collagen Types (e.g., Fibrillar (I, II, III, V, XI), Facit (IX, XII, XIV), Short chain (VIII, X), Basement membrane (IV), and Other (VI, VII, XIII); Elastin; DNA; RNA; Fibronectins and glycoproteins; Laminin-111 (Laminin-1); Laminin-211 (Laminin-2); Laminin-121 (Laminin-3); Laminin-221 (Laminin-4); Laminin-332/Laminin-3A32 (Laminin-5/Laminin-5A); Laminin-3B32 (Laminin-5B); Laminin-311/Laminin-3A11 (Laminin-6/Laminin-6A); Laminin-321/Laminin-3A21 (Laminin-7/Laminin-7A); Laminin-411 (Laminin-8); Laminin-421 (Laminin-9); Laminin-511 (Laminin-10); Laminin-521 (Laminin-11); Laminin-213 (Laminin-12); Laminin-423 (Laminin-14); Laminin-522; Laminin-523 (Laminin-15).

In accordance with certain embodiments of the invention, the custom formed extracellular matrix facilitates organization and arrangement of patient stem cells on the extracellular matrix scaffold. Moreover, the extracellular matrix scaffold enables differentiation of patient stem cells into requisite cells to form tissue. As noted above, methods in accordance with certain embodiments of the invention allows for custom formation of tissue grafts using the patient's own cells that are biocompatible with the specific patient, instead of relying on synthetically made tissues or donor tissues.

In accordance with certain embodiments of the invention, the methods disclosed herein allow for bioengineered patient tissues to be made for regenerative applications or to deliver therapeutic cells without need to rely on synthetic tissues, donor tissues, or bioengineered tissues made from donor tissues. For example, certain embodiments of the invention provide for methods of making a custom autograft using a patient's own stem cells and/or somatic cells. In accordance with certain embodiments of the invention, the present invention beneficially eliminates the need for a patient to take immunosuppressive drugs because the tissue is generated from the patient instead of a donor or synthetic material.

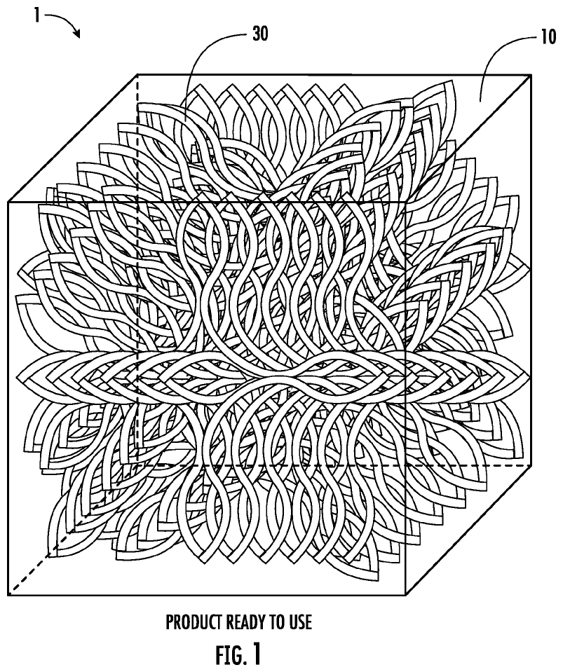

Certain embodiments according to the invention provide an acellular substrate (e.g., a scaffolding) comprising a three-dimensional (3D) macrostructure defined by a continuous matrix of extracellular matrix (ECM) material associated with a first cell type of interest and a network of microporous channels and/or chambers extending throughout the continuous matrix of ECM material associated with the first cell type of interest. The 3D macrostructure may comprise a top surface, a bottom surface, and a thickness defined by at least one side edge extending from the top surface to the bottom surface. FIG. 1, for instance, illustrates an acellular substrate 1 having a 3D macrostructure defined by a continuous matrix of ECM material 10 and a network of microporous channels and/or chambers 30 extending throughout the continuous matrix of ECM material. The continuous matrix of ECM material 10 in FIG. 1 is rendered transparent to illustrate the network of microporous channels and/or chambers 30 extending therethrough.

In accordance with certain embodiments of the invention, the acellular substrate may further comprise at least one interlocking-male component and/or at least one interlocking-female component, each of which may be used to interlock or otherwise join two or more individual acellular substrates together. By way of example, the at least one interlocking-male component (if present) may include a first interlocking-male component extending outwardly from the at least one side edge. The at least one side edge, for instance, may include a first side edge and a second side edge, and wherein the at least one interlocking-male component includes a first interlocking-male component extending outwardly from the first side edge and a second interlocking-male component extending outwardly from the second side edge. Additionally or alternatively, the at least one interlocking-male component includes a third interlocking-male component extending outwardly from the top surface. In accordance with certain embodiments of the invention, the acellular substrate may include a first interlocking-female component extending inwardly from the at least one side edge towards an interior portion of the 3D macrostructure. For example, the at least one side edge includes a third side edge and a fourth side edge, and wherein the at least one interlocking-female component includes a first interlocking-female component extending inwardly from the third side edge towards an interior portion of the 3D macrostructure and a second interlocking-female component extending inwardly from the fourth side edge towards an interior portion of the 3D macrostructure. In accordance with certain embodiments of the invention, the first side edge and the third side edge define a first pair of opposing side edges, while the second side edge and the fourth side edge define a second pair of opposing side edges. Additionally or alternatively, the at least one interlocking-female component includes a third interlocking-female component extending inwardly from the bottom surface towards an interior portion of the 3D macrostructure.

In accordance with certain embodiments of the invention, the 3D macrostructure with the exception of the at least one interlocking-male component and at least one interlocking-female component (if they are present) defines a cube, a square prism, or a triangular prism. In accordance with certain embodiments of the invention, the 3D macrostructure with the exception of the at least one interlocking-male component and at least one interlocking-female component (if they are present) defines a polygonal prism having from 3 to 12 side edges, such as at least about 3, 4, 5, 6, 7, and 8 side edges, and/or at most about any of the following: 12, 11, 10, 9, and 8 side edges.

In accordance with certain embodiments of the invention, the at least one side edge includes a first side edge, a second side edge, and an arcuate side edge located between and adjacent the first side edge and the second side edge. For example, the first side edge may include the at least one interlocking-male component extending outwardly from the first side edge and the second side edge may include the at least one interlocking-female component extending inwardly from the second side edge towards an interior portion of the 3D macrostructure. Additionally or alternatively, the at least one interlocking-male component includes a second interlocking-male component extending outwardly from the top surface. Additionally or alternatively, the at least one interlocking-female component includes a second interlocking-female component extending inwardly from the bottom surface towards an interior portion of the 3D macrostructure. By way of example only, the 3D macrostructure with the exception of the at least one interlocking-male component and at least one interlocking-female component defines a semi-cylinder, such as ⅛th of a cylinder to ½ of a cylinder, such as ⅛th, ¼th, ⅓rd, or ½ of a cylinder.

In accordance with certain embodiments of the invention, the top surface comprises a macroscopic surface area from about 0.25 cm² to about 25 cm², such as at least about any of the following: 0.25, 0.5, 75, 1, 1.5, 2, 5, 8, 10, and 12 cm², and/or about any of the following: 25, 22, 20, 18, 15, and 12 cm². Additionally or alternatively, the bottom surface comprises a macroscopic surface area from about 0.25 cm² to about 25 cm², such as at least about any of the following: 0.25, 0.5, 0.75, 1, 1.5, 2, 5, 8, 10, and 12 cm², and/or about any of the following: 25, 22, 20, 18, 15, and 12 cm². Additionally or alternatively, the thickness of the 3D macrostructure is from about 0.5 cm to about 3 cm, such as at least about any of the following: 0.5, 0.75, 1, 1.25, and 1.5 cm, and/or at most about any of the following: 3, 2.5, 2, and 1.5 cm. Additionally or alternatively, each of the at least one interlocking-female component is configured to receive a corresponding at least one interlocking-male component of a second acellular substrate.

In accordance with certain embodiments of the invention, the network of microporous channels and/or chambers extending throughout the continuous matrix of ECM material may have an average diameter comprises from about 100 to about 800 microns, such as at least about any of the following: 100, 120, 150, 180, 200, 220, and 250 microns, and/or at most about any of the following: 800, 780, 750, 720, 700, 680, 650, 620, 600, 580, 550, 520, 500, 480, 450, 420, 400, 380, 350, 320, 300, 280, and 250 microns.

In another aspect, certain embodiments according to the invention provide a method of forming an acellular substrate, comprising: (i) forming or placing a network of microstrands and/or micropods comprising a degradable hydrogel material within a mold; (ii) seeding the network of microstrands and/or micropods by adding an initial culture media including cells of a cell type of interest into the mold housing the network of microstrands and/or micropods; (iii) feeding the cells by perfusing fresh culture media through the mold to provide cells with nutrients until a tissue has grown and expanded to fill the mold; (iv) performing a decellularization operation on the tissue located in the mold forming a continuous matrix of ECM material associated with the cell type of interest; and (v) forming a network of microporous channels and/or chambers extending throughout the continuous matrix of ECM material associated with the cell type of interest by degrading and removing the network of microstrands and/or micropods to provide the acellular substrate.

In accordance with certain embodiments of the invention, wherein forming or placing a network of microstrands and/or micropods comprises performing an additive manufacturing technique, such as 3D printing of digital light synthesis printing. Additionally or alternatively,
a structure of the network of microstrands and/or micropods is selected based on a cell morphology of the cell type of interest. For example, the cell morphology of the cell type of interest has a target matrix structure and a target microporous network of channels and/or chambers, wherein the structure of the network of microstrands and/or micropods mimics or is identical to the target microporous network of channels and/or chambers. Additionally or alternatively, the network of microstrands and/or micropods has an average diameter comprises from about 100 to about 800 microns, such as at least about any of the following: 100, 120, 150, 180, 200, 220, and 250 microns, and/or at most about any of the following: 800, 780, 750, 720, 700, 680, 650, 620, 600, 580, 550, 520, 500, 480, 450, 420, 400, 380, 350, 320, 300, 280, and 250 microns.

In accordance with certain embodiments of the invention, the network of microstrands and/or micropods comprises a selectably degradable hydrogel material comprising one or more degradable polymers, such as one or more biopolymers derived from a living organism. For example, the one or more biopolymers derived from a living organism may comprise a polynucleotide, polysaccharide, polypeptide, or any combination thereof. In accordance with certain embodiments of the invention, the one or more biopolymers comprises collagen, gelatin, laminin, alginate, glycosaminoglycans, oligonucleotides (e.g., DNA, RNA), carbohydrates, lipids, cellulose, alginate, and proteins that can be gently and degraded, such as with the use of protein specific enzymes, ionic solvents, neutral detergents, weak acids, and peroxides to disrupt the biopolymer chains. In accordance with certain embodiments of the invention, the one or more biopolymers comprises degradable monomers comprising esters, such as hydroxybutyrate, lactic acid, glycolic acid, and caprolactone; anhydrides, such as adipic acid, and sebacic acid; saccharides, such as cellulose, alginate, pectin, dextrin, chitosan, hyaluronan, Chondroitin sulfate, and heparin; proteins; nucleotides (DNA, RNA); peptides, such as collagen, gelatin, silk, and fibrin; urethanes; phosphates; carbonates; and vinyl chlorides. Additionally or alternatively, the selectably degradable hydrogel material further comprises a synthetic polymer, such as a polyester, a polyanhydride, a polycarbonate, a polyurethane, a polyphosphate or combinations thereof. In accordance with certain embodiments of the invention, the selectably degradable hydrogel material comprises from 0 to 25% by weight of a synthetic polymer, such as at least about any of the following: 0, 1, 3, 5, 8, and 10% by weight of a synthetic polymer, and/or at most about any of the following: 25, 20, 15, and 12% by weight of a synthetic polymer.

In accordance with certain embodiments of the invention, the step of performing the decellularization operation on the tissue located in the mold comprises treating the tissue with a detergent followed by dialyzing the continuous matrix of ECM material associated with the cell type of interest. In this regard, the primary cells may be selectably removed while preserving the structure of the ECM material. After decellularization, the method may comprise a step of lyophilizing the continuous matrix of ECM material associated with the cell type of interest. The method may further comprise a step of removing the acellular substrate from the mold and/or a step of sterilizing the acellular substrate, such as by e-beam or gamma irradiation operations.

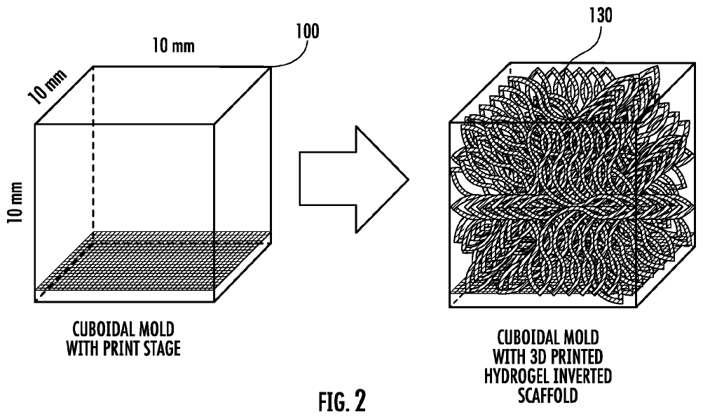

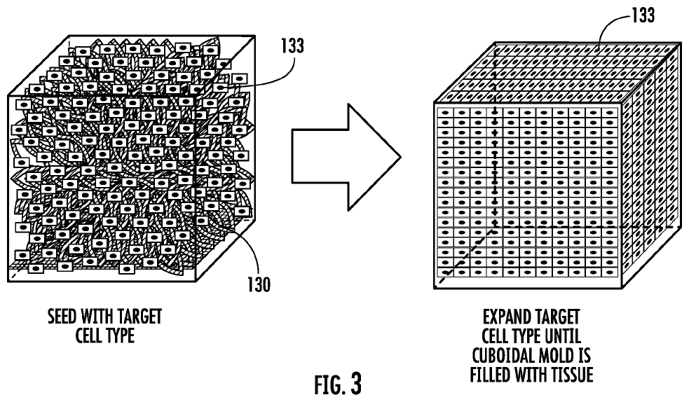

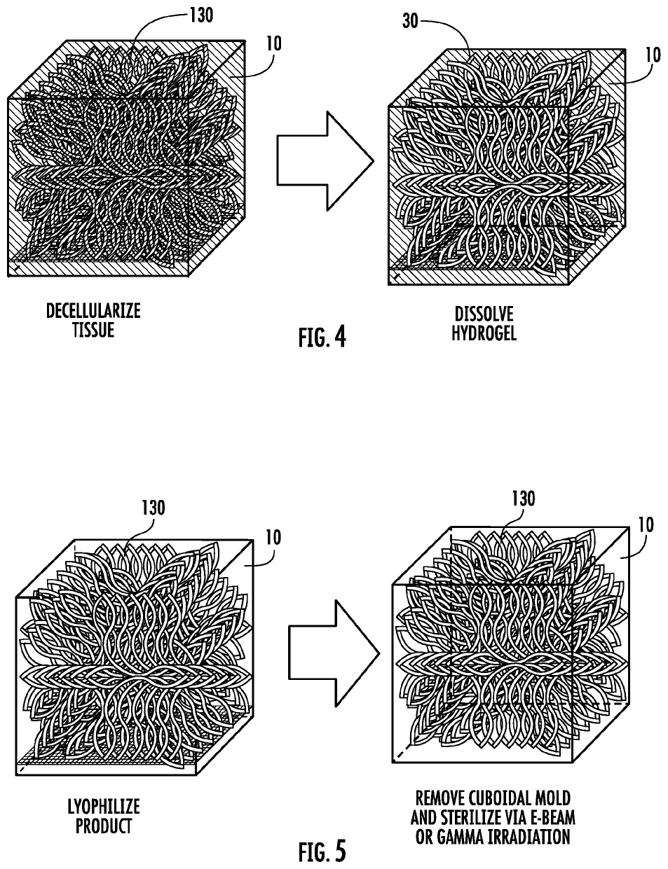

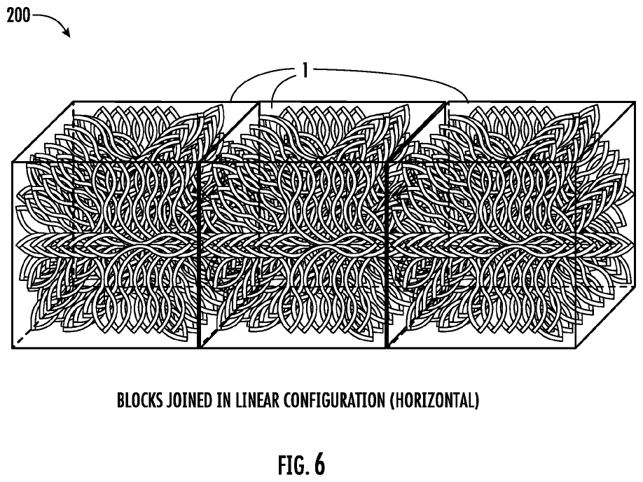

FIGS. 2-5 illustrate a general method for making an acellular substrate. FIG. 2 illustrates a cuboidal mold 100 having a network of microstrands and/or micropods 130 3D printed therein or disposed therein. The network of microstrands and/or micropods 130 may be formed from a degradable or dissolvable hydrogel material, such as those described and disclosed herein. FIG. 3 illustrates the seeding of a cell type of interest 33 onto the network of microchannels and/or micropods 130, and growth of the extracellular matrix of the cell type of interest to fill the cuboidal mold, which may include at least one interlocking-male component and/or at least one interlocking-female component (not shown). After the cuboidal mold 100 is filled with the extracellular matrix material, a decellularized tissue 10 is produced as illustrated in FIG. 4 as well as a subsequent degradation or dissolution of the hydrogel material (e.g., the network of microstrands and/or micropods) 30 to form an intermediate acellular substrate. FIG. 5 illustrates the intermediate acellular substrate being lyophilized and subsequently removed from the cuboidal mold followed by sterilization. FIG. 1 illustrates the resulting acellular matrix formed in accordance with certain embodiments of the invention.

In another aspect, certain embodiments according to the invention provide a method of forming a personalized graft, comprising: (i) providing or forming one or more acellular substrates, such as those described and disclosed herein, wherein the first cell type of interest is associated with a patient's tissue having an anomaly, such a particular organ tissue; (ii) seeding the one or more acellular substrates with healthy native cells associated with the patients tissue having an anomaly; and (iii) feeding the healthy native cells with a culture media, and allowing the healthy native cells to propagate throughout the network of microporous channels and/or chambers of the acellular substrate forming the personalized graft. For example, the cells of the first cell type are removed from the ECM material, and the patient's own cells may be added to the acellular substrate designed for them. This means when a patient has an anomaly in a tissue, a custom mold (e.g., a 3D network of microstrands and/or micropods based on the tissue morphology of the tissue in question and formed from a hydrogel material) can be created to fix the anomaly, and create a custom scaffold (e.g., dissolution or degradation of the hydrogel material forming the 3D network of microstrands and/or micropods) personalized graft for the patient. As such, methods in accordance with certain embodiments of the invention provide for the creation of custom scaffolds from a patient's own anatomy to form grafts from the application of a patient's own cells.

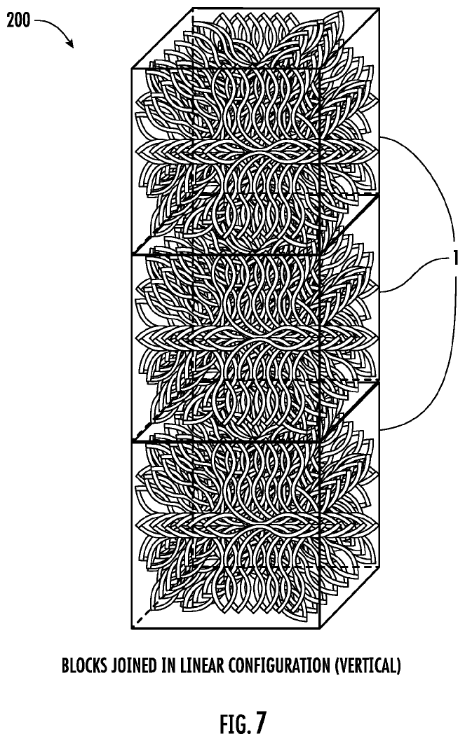

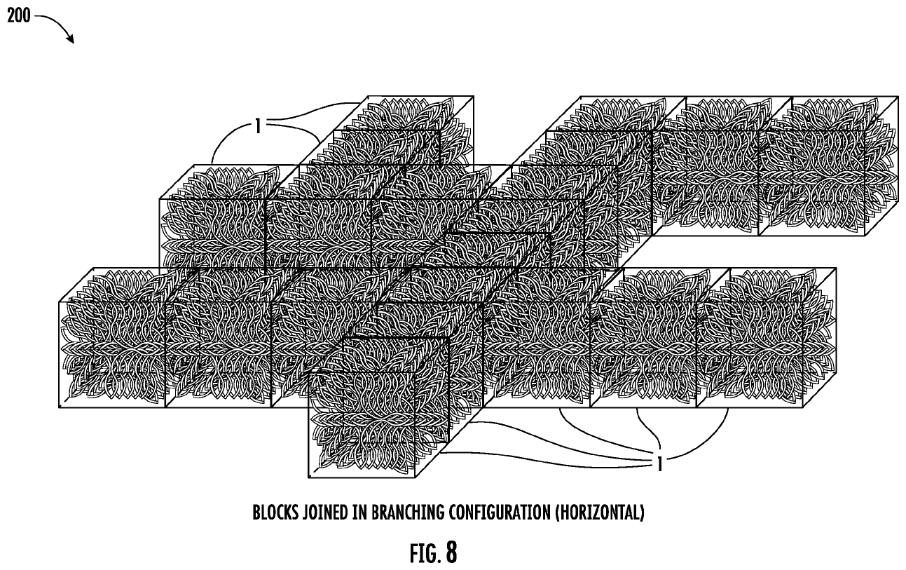

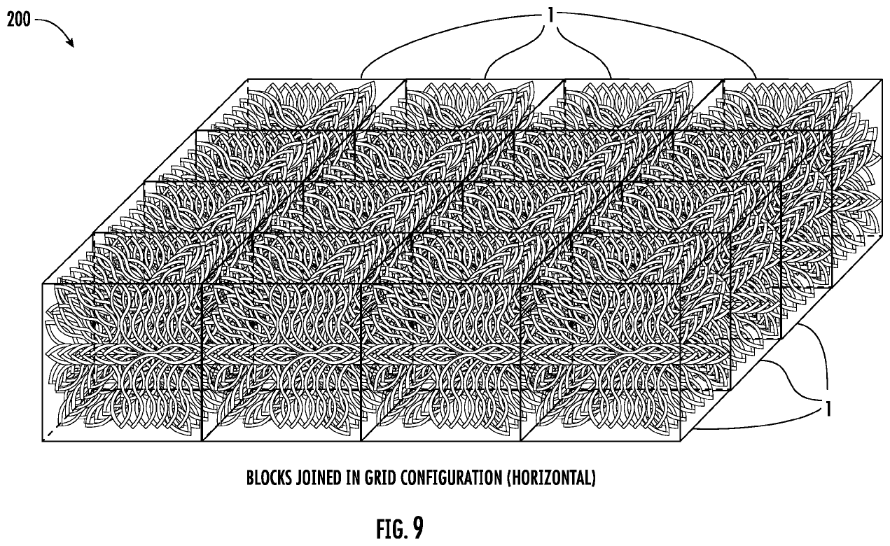

In accordance with certain embodiments of the invention, the one or more acellular substrates comprises a first acellular substrate and a second acellular substrate, wherein the first acellular substrate and the second acellular substrate are the same. For example, the first acellular substrate may be joined to the second acellular substrate such that a multi-acellular scaffolding is provided. The method may include allowing the healthy native cells to propagate throughout an aggregate network of microporous channels and/or chambers of the multi-acellular scaffolding forming the personalized graft. In accordance with certain embodiments of the invention, the one or more acellular substrates comprises from at least 2 acellular substrates joined together to define a multi-acellular scaffolding, or at least about any of the following: 2, 3, 5, 10, 20, 30, 40, 50, 60, 70, 80, 90, and 100 acellular substrates. FIGS. 6-9 illustrate non-limiting examples of different configurations 200 of a plurality of joined acellular substrates 1 in accordance with certain embodiments of the invention. As illustrated by FIGS. 6-9, the acellular substrates may be interlocked or otherwise joined to together in a multitude of different 3D geometric shapes.

### Examples

The present disclosure is further illustrated by the following examples, which in no way should be construed as being limiting. That is, the specific features described in the following examples are merely illustrative and not limiting.

A unique closed system has been designed where liquids are applied and aspirated as needed. Inverse hydrogel molds (e.g., the network of microstrands and micropods) were first deposited into cuboidal molds. Next, sterile saline was applied twice for a duration of 5 minutes each. Afterward, cell media containing 1 million cells per mL are applied to each mold. Media was fully exchanged via perfusion every 24 hours. After a minimum of 7 days, cell media was removed, and the molds are washed with sterile saline. Sodium dodecyl sulfate (SDS) and sodium palmate (SP) are applied to each mold for up to 72 hours with gentle agitation. The SDS and SP were aspirated, and the ECM was dialyzed with sterile deionized water (DI) for up to 72 hours. The ECM was then cycled between −170° C. and 37° C. over six days. SDS and SP was applied to each mold for up to 72 hours with gentle agitation. The SDS and SP were aspirated, and the ECM was dialyzed with sterile DI water for up to 72 hours. The inverse mold (e.g., the network of microstrands and micropods) was then dissolved via photo lysis. ECM was washed with sterile saline and 10% antibiotics 3 times for 60 minute durations under UV light. ECM was washed with sterile saline. ECM was then packaged and lyophilized for storage.

These and other modifications and variations to the invention may be practiced by those of ordinary skill in the art without departing from the spirit and scope of the invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

1-40. (canceled)

41. An acellular substrate, comprising:
a three-dimensional (3D) macrostructure defined by a continuous matrix of extracellular matrix (ECM) material associated with a first cell type of interest and a network of microporous channels and/or chambers extending throughout the continuous matrix of ECM material associated with the first cell type of interest, and wherein the 3D macrostructure comprises a top surface, a bottom surface, and a thickness defined by at least one side edge extending from the top surface to the bottom surface.

42. The acellular substrate of claim 41, wherein the acellular substrate further comprises at least one interlocking-male component and at least one interlocking-female component, wherein the at least one interlocking-male component includes a first interlocking-male component extending outwardly from the at least one side edge and/or wherein the at least one interlocking-female component includes a first interlocking-female component extending inwardly from the at least one side edge towards an interior portion of the 3D macrostructure.

43. The acellular substrate of claim 42, wherein the at least one side edge includes a first side edge and a second side edge, and wherein the at least one interlocking-male component includes a first interlocking-male component extending outwardly from the first side edge and a second interlocking-male component extending outwardly from the second side edge.

44. The acellular substrate according to claim 43, wherein the at least one side edge includes a third side edge and a fourth side edge, and wherein the at least one interlocking-female component includes a first interlocking-female component extending inwardly from the third side edge towards an interior portion of the 3D macrostructure and a second interlocking-female component extending inwardly from the fourth side edge towards an interior portion of the 3D macrostructure, and optionally wherein the first side edge and the third side edge define a first pair of opposing side edges, and the second side edge and the fourth side edge define a second pair of opposing side edges.

45. The acellular substrate according to claim 41, wherein (i) the top surface comprises a macroscopic surface area from about 0.25 cm² to about 25 cm²; (ii) the bottom surface comprises a macroscopic surface area from about 0.25 cm² to about 25 cm²; (iii) the thickness of the 3D macrostructure is from about 0.5 cm to about 3 cm; or (iv) any combination of (i), (ii), and (iii).

46. The acellular substrate according to claim 42, wherein each of the at least one interlocking-female component is configured to receive a corresponding at least one interlocking-male component of a second acellular substrate.

47. The acellular substrate according to claim 41, wherein (i) the network of microporous channels and/or chambers extending throughout the continuous matrix of ECM material has an average diameter comprises from about 100 to about 800 microns; (ii) the network of microporous channels and/or chambers extending throughout the continuous matrix of ECM material has comprises at least about 40% by volume of the 3D macrostructure; or (iii) both (i) and (ii).

48. A method of forming an acellular substrate, comprising:
(i) forming or placing a network of microstrands and/or micropods comprising a degradable hydrogel material within a mold;
(ii) seeding the network of microstrands and/or micropods by adding an initial culture media including cells of a cell type of interest into the mold housing the network of microstrands and/or micropods;
(iii) feeding the cells by perfusing fresh culture media through the mold to provide cells with nutrients until a tissue has grown and expanded to fill the mold;
(iv) performing a decellularization operation on the tissue located in the mold forming a continuous matrix of ECM material associated with the cell type of interest;
(v) forming a network of microporous channels and/or chambers extending throughout the continuous matrix of ECM material associated with the cell type of interest by degrading and removing the network of microstrands and/or micropods to provide the acellular substrate.

49. The method of claim 48, wherein forming or placing a network of microstrands and/or micropods comprises performing an additive manufacturing technique, such as 3D printing of digital light synthesis printing.

50. The method according to claim 48, wherein a structure of the network of microstrands and/or micropods is selected based on a cell morphology of the cell type of interest, in which the cell morphology has a target matrix structure and a target microporous network of channels and/or chambers; wherein the structure of the network of microstrands and/or micropods mimic or are identical to the target microporous network of channels and/or chambers.

51. The methods according to claim 48, wherein the network of microstrands and/or micropods has an average diameter comprises from about 100 to about 800 microns.

52. The methods according to claim 48, wherein the network of microstrands and/or micropods comprises a selectably degradable hydrogel material comprising one or more degradable polymers, such as one or more biopolymers derived from a living organism.

53. The method according to claim 52, wherein the one or more biopolymers comprises collagen, gelatin, laminin, alginate, glycosaminoglycans, oligonucleotides, carbohydrates, lipids, cellulose, alginate, and proteins that are degradable with the use of protein specific enzymes, ionic solvents, neutral detergents, weak acids, and/or peroxides to disrupt biopolymer chains thereof.

54. The method according to claim 52, wherein the selectably degradable hydrogel material further comprises a synthetic polymer, such as a polyester, a polyanhydride, a polycarbonate, a polyurethane, a polyphosphate or combinations thereof.

55. The method according to claim 48, wherein performing the decellularization operation on the tissue located in the mold comprises treating the tissue with a detergent followed by dialyzing the continuous matrix of ECM material associated with the cell type of interest.

56. The method according to claim 48, further comprising (i) a step of lyophilizing the continuous matrix of ECM material associated with the cell type of interest; (ii) a step of sterilizing the acellular substrate, such as by e-beam or gamma irradiation operations; (iii) a step of removing the acellular substrate from the mold; or (iv) any combination of (i), (ii), and (iii).

57. A method of forming a personalized graft, comprising:
(i) providing or forming one or more acellular substrates according to claim 1, wherein the first cell type of interest is associated with a patient's tissue having an anomaly, such a particular organ tissue;
(ii) seeding the one or more acellular substrates with healthy native cells associated with the patients tissue having an anomaly; and
(iii) feeding the healthy native cells with a culture media, and allowing the healthy native cells to propagate throughout the network of microporous channels and/or chambers of the acellular substrate forming the personalized graft.

58. The method of claim 57, wherein the one or more acellular substrates comprises a first acellular substrate and a second acellular substrate, wherein the first acellular substrate and the second acellular substrate are the same.

59. The method of claim 57, wherein the first acellular substrate is joined to the second acellular substrate such that a multi-acellular scaffolding is provided, and the allowing the healthy native cells to propagate throughout an aggregate network of microporous channels and/or chambers of the multi-acellular scaffolding forming the personalized graft.

60. The method according to claim 57, wherein the one or more acellular substrates comprises from at least 2 acellular substrates joined together to define a multi-acellular scaffolding.
